(19) United States
(12) Patent Application Publication
(10) Pub. No.: US 2011/0212171 A1
(43) Pub. Date: Sep. 1, 2011

(54) TASTE MASKED TOPIRAMATE COMPOSITION AND AN ORALLY DISINTEGRATING TABLET COMPRISING THE SAME

(75) Inventors: Gopi M. Venkatesh, Vandalia, OH (US); Troy M. Harmon, Lansdale, PA (US)

(73) Assignees: EURAND, INC., Vandalia, OH (US); EURAND PHARMACEUTICALS, INC., Yardly, PA (US)

(21) Appl. No.: 12/986,457

(22) Filed: Jan. 7, 2011

Related U.S. Application Data
(60) Provisional application No. 61/293,455, filed on Jan. 8, 2010.

(51) Int. Cl. A61K 9/20; A61K 9/50; A61K 31/70; B29C 43/00

(52) U.S. Cl. 424/464; 424/490; 514/23; 424/497; 424/494; 264/109

(57) ABSTRACT

In various embodiments, the present invention is directed to a taste masked pharmaceutical composition comprising a therapeutically effective amount of taste masked sulfamate-substituted monosaccharide particles comprising a sulfamate-substituted monosaccharide or a pharmaceutically acceptable salt or derivative thereof that are coated with one or more taste-masking layers, and optionally one or more of taste-masked neltrexone, 5-HT₃ receptor antagonist, phentermine, and vitamin B-12. The present invention relates to methods of making the taste masked and ODT compositions, and methods of using the compositions for treating a patient subject to an epileptic condition, migraines, dysphagia, achieving/maintaining weight loss, or alcoholism or drug addiction.

### CROSS REFERENCE TO RELATED APPLICATIONS

This application claims the benefit of U.S. Provisional Application No. 61/293,455, filed Jan. 8, 2010, which is herein incorporated by reference in its entirety for all purposes.

### BACKGROUND OF THE INVENTION

Dysphagia, or difficulty in swallowing due to fear of choking, is common among all age groups. For example, it is observed in about 35% of the general population, as well as an additional 30-40% of elderly institutionalized patients and 18-22% of all persons in long-term care facilities, many of whom are required to consume medications on a regular basis to maintain their quality of life, may suffer from dysphagia. This may lead to poor compliance or even non-compliance with treatments comprising oral medications, and thus has a negative impact on the efficacy of such treatments.

The primary treatment objectives for patients with epilepsy in mono- or adjunct therapy are maintenance of adequate anti-epileptic drug levels and prevention of additional seizures. In either case, compliance with a prescribed drug dosage regimen is essential for the maintenance of a therapeutic drug concentration in the blood.

Topiramate, 2,3:4,5-bis-O-isopropylidene-β-D-fructopyranose sulfamate, has the following structural formula (I):

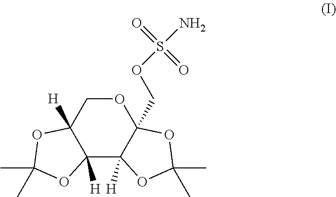

Topiramate is the active ingredient in the marketed product TOPAMAX® which is used as an anti-epileptic drug. Topiramate is widely prescribed as an initial monotherapy in patients 10 years of age and older with partial onset or primary generalized tonic-clonic seizures. Topiramate tablets or capsules are also indicated as adjunctive therapy for adults and pediatric patients ages 2-16 years with partial onset seizures, or primary generalized tonic-clonic seizures, and in patients 2 years of age and older with seizures associated with Lennox-Gastaut syndrome. Topiramate is also indicated for adults for the prophylaxis of migraine headache. Tablets of TOPAMAX® are available as 25 mg, 50 mg, 100 mg, and 200 mg round tablets for oral administration or TOPAMAX® capsules available as 15 mg and 25 mg sprinkle capsules for oral administration as whole capsules or opened and sprinkled onto soft food. However, topiramate has a bitter taste.

ODT formulations should be palatable, e.g., they should have acceptable organoleptic properties such as good taste and mouthfeel to maintain patient compliance or adherence to the dosing regimen, because ODT tablets are designed to disintegrate in the mouth of the patient. ODT compositions should also exhibit acceptable pharmacokinetic and bioavailability characteristics to provide a desired therapeutic effect comparable to a non-ODT dosage from (e.g., a conventional tablet, capsule, etc.), to avoid expensive in vivo efficacy testing and validation relative to such conventional dosage forms. For a bitter tasting drug, an ODT formulation generally requires the application of a taste-masking layer to drug-containing particles to improve the organoleptic characteristics of the ODT formulation, such that the taste and mouthfeel of the dissolved ODT remain acceptable to a subject until the contents of the dosage form are swallowed, typically without water or other fluids. However, taste-masking can inhibit or delay drug release, thereby providing unacceptable pharmacokinetic properties. Conversely, certain components of the formulation may promote rapid release, and thus may result in undesirable taste or mouthfeel properties.

A drug product is also generally required to not only meet certain bioequivalence criteria, but may also need to meet certain dissolution requirements in accordance with local regulatory guidance, e.g., “Guideline for Bioequivalence Studies of Generic Products” (Japanese Government regulatory requirements). For example, if the dissolution rate of a test product and that of a reference product exhibit “a specific, marked difference in a dissolution medium at pH=6.8 or between pH 3.0 and 6.8 for a basic drug substance,” an expensive, time consuming, and/or ‘not easy to recruit’ bioequivalence study in subjects with low gastric acidity may need to be performed.

The term “marked difference” may mean either (1) at the time point when the mean dissolution rate of the product with higher dissolution reaches 80%, the mean dissolution rate of the other product does not reach 50%, or (2) when the mean dissolution rate of both products does not reach 80% within the prescribed testing time and the mean dissolution rate of the other product with the slower dissolution does not reach 60% of the mean dissolution rate of the other product at the end of the prescribed testing time.

Accordingly, an acceptable ODT formulation should balance the above-described contradictory characteristics in order to provide a palatable (e.g., taste-masked), fast disintegrating composition with acceptable in vitro and or in vivo dissolution profiles as well as acceptable pharmacokinetics.

Currently there is no ODT formulation comprising topiramate microparticles meeting the following specifications:
topiramate containing microparticles that are not only effectively taste-masked but which also exhibit a dissolution profile meeting regulatory requirements;
ODT comprises rapidly-dispersing granules so as to rapidly disintegrate on contact with saliva in the oral cavity forming a smooth, easy-to-swallow suspension containing taste-masked topiramate containing microparticles;
topiramate containing particles having an average particle diameter of not more than 400 μm to provide smooth mouthfeel and leave no aftertaste (i.e., little or minimal drug release with a non-gritty or non-chalky taste) before being swallowed;
provides for rapid, substantially-complete release of topiramate upon arrival in the stomach in order to be bioequivalent to a Reference Listed Drug (RLD) (i.e., immediate-release reference-listed-drug product, TOPAMAX®).
ODT formulation that exhibits acceptable tablet hardness and friability to be suitable for packaging in high density polyethylene (HDPE) bottles or blisters for transportation and commercial distribution.

### SUMMARY OF THE INVENTION

The present disclosure relates to taste masked pharmaceutical compositions. Specifically, the present disclosure relates to a taste masked pharmaceutical composition comprising a therapeutically effective amount of taste masked sulfamate-substituted monosaccharide particles, comprising a sulfamate-substituted monosaccharide or a pharmaceutically acceptable salt or derivative thereof, and wherein the microparticles are coated with one or more taste-masking layers to taste mask the sulfamate-substituted monosaccharide; wherein said taste-masking layer comprises at least one water-insoluble polymer. The present disclosure also relates to an immediate release (IR) orally disintegrating tablet (ODT) comprising a therapeutically effective amount of a population of particles wherein each particle comprises (i) microparticles comprising topiramate or a sulfamate-substituted monosaccharide, or a pharmaceutically acceptable salt or derivative thereof, coated with one or more taste-masking layers, and (ii) rapidly dispersing microgranules comprising at least one disintegrant and at least one sugar alcohol and/or at least one saccharide; wherein the taste/taste-masking layer comprises a water-insoluble polymer. The present disclosure also relates to methods of making the taste masked and ODT compositions, and methods of using the present compositions for treating a patient subject to an epileptic condition, seizures or migraine, and/or for achieving/maintaining weight loss, and/or managing or treating alcoholism and/or drug addiction.

In one embodiment, the present invention is directed to a taste masked pharmaceutical composition comprising a therapeutically effective amount of taste masked sulfamate-substituted monosaccharide particles comprising a sulfamate-substituted monosaccharide or a pharmaceutically acceptable salt or derivative thereof, wherein said particles are coated with one or more taste-masking layers to taste mask the sulfamate-substituted monosaccharide; wherein said taste-masking layer comprises at least one water-insoluble polymer. In another embodiment, the invention is directed to an orally disintegrating tablet (ODT), comprising a therapeutically effective amount of a population of particles wherein each particle comprises a microparticle comprising topiramate, a sulfamate-substituted monosaccharide, or a pharmaceutically acceptable salt or derivative thereof, wherein said microparticles are coated with a taste-masking layer, rapidly dispersing microgranules comprising at least one disintegrant, and at least one sugar alcohol and/or at least one saccharide; and wherein said taste-masking layer comprises a water-insoluble polymer. In still another embodiment, the present invention relates to methods of making the taste masked and ODT compositions. In another embodiment, the present invention relates to methods for using the present compositions for treating a patient subject to an epileptic condition, treating a patient to induce or maintain weight loss, or treating a patient suffering from migraines. These and other embodiments will be explained in detail below.

### DETAILED DESCRIPTION OF THE INVENTION

The following description includes information that may be useful in understanding the invention.

### DEFINITIONS

As used above, and throughout the description of the invention, the following terms, unless otherwise indicated, shall be understood to have the following meanings:

The term “drug”, “active”, active ingredient, or “active pharmaceutical ingredient” as used herein includes any pharmaceutically acceptable and therapeutically effective compound, pharmaceutically acceptable salts, stereoisomers and mixtures of stereoisomers, solvates (including hydrates), and/or derivatives thereof). One suitable drug according to the present invention is topiramate.

The terms “derivative,” “derivatives” or “derivatives thereof” as used herein includes chemical compounds which are related to topiramate. For example, in certain embodiments, derivatives of topiramate and/or a sulfamate-substituted monosaccharide may include compounds of formula I below:

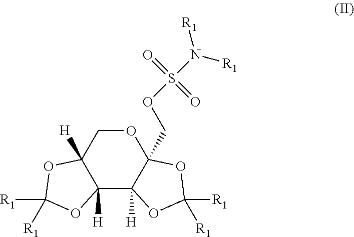

wherein each instance of R₁ is independently selected from the group consisting of: H, hydroxy, cyano, nitrito, F, Cl, Br, I, triflate, mesylate, tosylate, (C₁-C₆)alkyl, hydroxy(C₁-C₆)alkyl, (C₁-C₆)alkanoyl, aryl (C₁-C₆)alkyl. In certain embodiments, R₁ is H or methyl.

The term “salts” refers to the product formed by the reaction of a suitable inorganic or organic acid with the “free base” form of the drug. Suitable acids include those having sufficient acidity to form a stable salt, for example acids with low toxicity, such as the salts approved for use in humans or animals. Non-limiting examples of acids that may be used to form salts of topiramate include inorganic acids, e.g., HF, HCl, HBr, HI, H₂SO₄, H₃PO₄; non-limiting examples of organic acids include organic sulfonic acids, such as C₆₋₁₆ aryl sulfonic acids, C₆₋₁₆ heteroaryl sulfonic acids or C₁₋₁₆ alkyl sulfonic acids—e.g., phenyl, α-naphthyl, β-naphthyl, (S)-camphor, methyl, ethyl, n-propyl, i-propyl, n-butyl, s-butyl, i-butyl, t-butyl, pentyl and hexyl sulfonic acids; non-limiting examples of organic acids includes carboxylic acids such as C₁₋₁₆ alkyl, C₆₋₁₆ aryl carboxylic acids and C₄₋₁₆ heteroaryl carboxylic acids, e.g., acetic, glycolic, lactic, pyruvic, malonic, glutaric, tartaric, citric, fumaric, succinic, malic, maleic, hydroxymaleic, benzoic, hydroxybenzoic, phenylacetic, cinnamic, salicylic and 2-phenoxybenzoic acids; non-limiting examples of organic acids include amino acids, e.g. the naturally-occurring amino acids, lysine, arginine, glutamic acid, glycine, serine, threonine, alanine, isoleucine, leucine, etc. Other suitable salts can be found in, e.g., S. M. Birge et al., J. Pharm. Sci., 1977, 66, pp. 1-19 (herein incorporated by reference for all purposes). In most embodiments, “salts” refers to salts that are biologically compatible or pharmaceutically acceptable or non-toxic, particularly for mammalian cells. The salts of drugs useful in the invention may be crystalline or amorphous, or mixtures of different crystalline forms and/or mixtures of crystalline and amorphous forms.

The term “prodrug” means a form of the compounds of formula I and/or II suitable for administration to a patient without undue toxicity, irritation, allergic response, and the like, and effective for their intended use, including ketal, ester and zwitterionic forms. A prodrug is transformed in vivo to yield a compound of formula I and/or formula II, for example by hydrolysis in blood. A thorough discussion is provided in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987, both of which are incorporated herein by reference.

The terms “orally disintegrating tablet”, “orally dispersible tablet”, or “ODT” refer to a solid dosage form of the invention, which disintegrates rapidly in the oral cavity of a patient after administration, without the need for chewing. The rate of disintegration can vary, but is faster than the rate of disintegration of conventional solid dosage forms (e.g., tablets, capsules, etc.) which are intended to be swallowed immediately after administration, or faster than the rate of disintegration of chewable solid dosage forms, when tested as described herein (e.g., the USP <701> test method). ODT compositions of the invention can contain pharmaceutically acceptable ingredients which swell, dissolve or otherwise facilitating the disintegration or dissolution of the ODT composition.

The term “unit dose” refers to a pharmaceutical composition containing an amount of drug intended to be administered to a patient in a single dose.

The term “about” used herein in reference to a numerical quantity includes the noted numerical quantity, as well as values near the numerical quantity. For example, “about 60 second” includes 60 seconds, exactly, as well as values close to 60 seconds (e.g., 50 seconds, 55 seconds, 59 seconds, 61 seconds, 65 seconds, 70 seconds, etc.). In certain cases, the term “about [a numerical value]” may be understood to mean within a certain percent of the numerical value. Thus, “about 100” can include a range of up to 15% around a value of 100 (e.g., from 85-115, inclusive), Similarly, the term about may include a range of up to 14%, 13%, 12%, 11%, 10%, 9%, 8%, 7%, 6%, 5%, 4%, 3%, 2%, 1% or less than 1% around a numerical value.

The term “substantially disintegrates” in reference to the ODT tablet compositions of the invention means the disintegration of the ODT tablet largely into its constituent particles or microparticles which were previously compressed into tablets. Substantially disintegrates means at least about 50%, at least about 60%, at least about 70%, at least about 80%, at least about 90%, or about 100% of the ODT tablet has disintegrated into its constituent particles. Similarly, the term “substantially dissolves” in reference to the ODT tablet compositions of the invention means that the percentage of the active pharmaceutical ingredient released or dissolved from the ODT tablet is at least about 50%, at least about 60%, at least about 70%, at least about 80%, at least about 90%, or about 100% of the topiramate present in the ODT composition. While describing the characteristics or properties of one of the product components, terms such as “topiramate-containing particles” or “percentage release from topiramate-containing particles” are used; however, these terms are equally applicable to other components of the group consisting of a sulfamate-substituted monosaccharides. The term “particle”, “microparticle”, “granule” and “microgranule” may be used interchangeably herein to refer to a particle with a mean particle size of not more than 400 μm; and in some embodiments of not more than 300 μm, irrespective of the composition of the particle. The term “Microcaps®” refers specifically to taste-masked drug-containing particles with a mean particle size of not more than 400 p.m. The term “taste-masking” refers to a process of masking an undesirable taste (e.g., bitterness) of a drug. This is achieved by coating drug-containing particles with a sufficiently thick barrier layer. Unless indicated otherwise, all percentages and ratios are calculated by weight. Unless indicated otherwise, all percentages and ratios are calculated based on the total composition. The microparticles herein can be described as primary particles or secondary particles. Primary particles are unagglomerated, whereas secondary particles are agglomerated primary particles. Thus, primary particles are generally smaller than secondary particles.

One embodiment of the invention is directed to an orally disintegrating tablet (ODT) composition comprising a therapeutically effective amount of a population of particles wherein each particle comprises topiramate coated with a taste-masking layer and rapidly dispersing microgranules.

Another embodiment of the invention is directed to a population of particles wherein each particle is a drug-layered bead comprising an inert core coated with a topiramate-containing layer.

In certain embodiments, the taste-masking layer comprises a water-insoluble taste-masking polymer or a water-insoluble taste-masking polymer in combination with a water-soluble or gastrosoluble polymer.

Yet another embodiment of the invention is directed to a method of preparing the present ODT composition comprising preparing microparticles comprising topiramate; coating the topiramate-containing microparticles with a taste-masking layer; preparing granules comprising a disintegrant in combination with a sugar alcohol and/or a saccharide (called rapidly dispersing granules or disintegrant-containing granules); mixing the topiramate-containing microparticles coated with a taste-masking layer with the disintegrant-containing granules and optionally other pharmaceutically acceptable ingredients; and compressing the mixture into the ODT composition.

In still another embodiment, the invention is directed to a method of treating patients with epilepsy (mono- or adjunct-therapy), migraine, and/or treating a patient to induce or maintain weight loss, comprising administering the ODT composition of the present invention.

The topiramate-containing particles may include crystalline topiramate, topiramate granulated with one or more pharmaceutically acceptable excipients (e.g., fillers, binders, etc.), inert cores layered beads with a topiramate-containing (prepared for example by coating in a fluid bed coater) or topiramate-containing pellets (prepared for example by controlled spheronization or powder layering in Vector's Granurex). For example, crystalline topiramate can be a primary particle having an average particle size ranging from about 1-300 μm, including about 1-50 μm, about 1-100 μm, about 1-150 μm, about 1-200 μm, about 1-250 μm, about 50-100 μm, about 50-150 μm, about 50-200 μm, about 50-250 μm, about 50-300 μm, about 100-150 μm, about 100-200 μm, about 150-200 μm, about 150-250 μm, about 150-300 μm, about 200-250 μm, about 200-300 μm, or about 250-300 μm.

When the topiramate-containing particles are granules or drug layered beads or Granurex pellets, the topiramate-containing particles comprise at least a film-forming binder. The film-forming binder can comprise any suitable binder used in forming the topiramate-containing particles. Non-limiting examples of suitable film-forming binders may include water-soluble, alcohol-soluble or acetone/water soluble binders, e.g. polyvinylpyrrolidone (PVP), corn starch, polyethylene oxide, polyethylene glycol, hydroxypropyl methylcellulose (HPMC), methylcellulose, or hydroxypropylcellulose (HPC). The amount of film-forming binder in the topiramate-containing particles can range from about 0.5% to about 10%, including about 0.5%-1%, about 0.5%-2%, about 0.5%-5%, about 0.5%-7%, about 1%-2%, about 1%-5%, about 1%-7%, about 1%-10%, about 2%-5%, about 2%-7%, about 2%-10%, about 5%-7%, about 5%-10%, and about 7%-10%.

The topiramate-containing particles described herein can also include other pharmaceutically acceptable ingredients, for example, fillers, diluents or other excipients. Non-limiting examples of other pharmaceutically acceptable ingredients for the drug-containing granules include, for example, mannitol, lactose, microcrystalline cellulose, potassium sulfate, calcium phosphate, modified starch, and mixtures thereof. The amount of other pharmaceutically acceptable ingredients (e.g. fillers, diluents or other excipients) in the topiramate-containing particles can range from about 5%-80%, including about 5%-70%, about 5%-60%, about 5%-50%, about 5%-40%, about 5%-30%, about 5%-20%, about 5%-15%, about 5%-10%, about 10%-70%, about 10%-60%, about 10%-50%, about 10%-40%, about 10%-30%, about 10%-20%, about 10%-15%, about 20%-70%, about 20%-60%, about 20%-50%, about 20%-40%, about 20%-30%, about 20%-25%, about 30%-70%, about 30%-60%, about 30%-50%, about 30%-40%, about 30%-35%, about 40%-70%, about 40%-60%, about 40%-50%, about 40%-45%, about 50%-70%, about 50%-60%, about 50%-55%, about 60%-70%, or about 60%-65%.

In another embodiment, the drug-containing cores described herein can be in the form of topiramate-layered beads or powder layered Granurex pellets comprising a core, e.g. a pharmaceutically acceptable sugar sphere or cellulose sphere (e.g., Celphere® from Asahi Kasei or Cellets from Glatt), coated with a topiramate-containing layer comprising topiramate and a polymeric binder. Suitable polymeric binders include any of those disclosed herein, for example starches, modified celluloses (e.g., hydroxypropylcellulose, hydroxypropyl methylcellulose (hypromellose), carboxymethylcellulose sodium), alginic acid, polyvinyl pyrrolidone (povidone), and mixtures thereof. The amount of topiramate in the topiramate layer, and the thickness of the topiramate layer can be modified to provide a therapeutically effective dose of topiramate. In one embodiment, the topiramate-containing layer comprises about 90%-99% topiramate and about 1% to about 10% of a polymeric binder.

The topiramate-containing particles of the ODT compositions described herein (e.g., crystalline topiramate, granulated topiramate, or topiramate-layered beads/pellets) are coated with a taste-masking layer. The taste masking layer may comprise a water-insoluble polymer, optionally in combination with a water-soluble or gastrosoluble or enterosoluble pore-former. These pore-formers may increase the release rate of the topiramate through the taste-masking layer. Water-soluble pore formers typically dissolve readily in water or saliva, whereas gastrosoluble pore formers are generally insoluble in water and saliva, but are readily soluble under acidic conditions (e.g., those present in the stomach of a human subject, typically less than about pH 5). In contrast, enterosoluble pore-forming polymers dissolve only in the intestinal region of the GI tract (e.g., under pH conditions found in the intestinal tract of a human subject, typically above a pH of about 4.5).

Non-limiting examples of suitable water-insoluble polymers may include, e.g., ethyl cellulose, polyvinyl acetate (PVA), cellulose acetate (CA), cellulose acetate butyrate (CAB), and methacrylate copolymers available under the tradename “EUDRAGIT” (such as Eudragit RL, Eudragit RS, Eudragit NE30D, etc.). Non-limiting examples of water-soluble pore-forming polymers may include, e.g., polyethylene glycol, povidone, hydroxypropylcellulose, methylcellulose, hypromellose, and mixtures thereof. Non-limiting examples of gastrosoluble pore-formers may include, e.g., calcium carbonate, magnesium citrate, magnesium hydroxide, and mixtures thereof. Non-limiting examples of gastrosoluble pore-forming polymers may include, e.g., Eudragit® E100/EPO (aminoalkyl methacrylate and neutral methacrylic acid ester), AEA® (polyvinylacetal diethylaminoacetate available from Sankyo Company Limited, Tokyo), and mixtures thereof. Non-limiting examples of enterosoluble pore-forming polymers may include, e.g., cellulose acetate phthalate, hypromellose phthalate, Eudragit® L100 or S100, and mixtures thereof. When a pore former is present in the taste-masking layer, the ratio of water-insoluble polymer to water-soluble, gastrosoluble or enterosoluble pore-former may vary from about 95/5 to about 50/50 by weight, or any other ratio therein. The amount of the taste-masking coating may range from about 5% to about 30% of the total weight of the taste-masked topiramate-containing particles, or about 5%-25%, about 5%-20%, about 5%-15%, about 5%-10%, about 10%-30%, about 10%-25%, about 10%-20%, about 10%-15%, about 15%-30%, about 50%-25%, about 15%-20%, about 20%-30%, about 20%-25%, or about 25%-30%, or any other value or range of values within the recited ranges.

The ODT compositions described herein include rapidly dispersing granules comprising a disintegrant and a sugar alcohol and/or a saccharide. Rapidly dispersing microgranules typically comprise a sugar alcohol such as mannitol and/or a saccharide such as lactose and a super disintegrant such as crospovidone. The sugar alcohol and/or saccharide and disintegrant are usually present in the rapidly dispersing microgranules at a ratio of from about 99:1 to about 90:10 (sugar alcohol and/or saccharide:disintegrant).

The disintegrant may include a “super-disintegrant.” Non-limiting examples of so-called super-disintegrants may include crospovidone (crosslinked PVP), sodium starch glycolate, crosslinked sodium carboxymethyl cellulose, low substituted hydroxypropylcellulose, and mixtures thereof. The amount of disintegrant in the rapidly dispersing granules can range from about 1%-10%, or about 5%-10% of the total weight of the rapidly dispersing granules, for example about 1%, about 2%, about 3%, about 4%, about 5%, about 6%, about 7%, about 8%, about 9%, or about 10% including all ranges, subranges and values therebetween.

Sugar alcohols are typically hydrogenated forms of carbohydrates in which a carbonyl group (e.g., an aldehyde or ketone) has been reduced to a primary or secondary hydroxyl group. Non-limiting examples of suitable sugar alcohols for inclusion in the rapidly dispersing granules of the present ODT compositions may include, e.g., arabitol, ISOMALT® (a disaccharide composed of the two sugars, glucose and mannitol), erythritol, glycerol, lactitol, mannitol, sorbitol, xylitol, maltitol, and mixtures thereof.

The term “saccharide” is generally synonymous with the term “sugars”, and includes monosaccharides such as glucose, fructose, lactose, and ribose; and disaccharides such as sucrose, lactose, maltose, trehalose, and cellobiose. Non-limiting examples of suitable saccharides for use on the compositions of the invention include e.g. lactose, sucrose, maltose, and mixtures thereof.

In yet another embodiment, the rapidly dispersing granules may comprise at least one disintegrant in combination with a sugar alcohol. In another embodiment, the rapidly dispersing granules may comprise at least one disintegrant in combination with a saccharide. In yet another embodiment, the disintegrant-containing granules may comprise at least one disintegrant in combination with a sugar alcohol and a saccharide.

The amount of sugar alcohol and/or saccharide in the rapidly dispersing granules may range from about 99%-90%, or about 95%-90% of the total weight of the rapidly dispersing granules, for example about 90%, about 91%, about 92%, about 93%, about 94%, about 95%, about 96%, about 97%, about 98%, or about 99%, including all ranges, subranges and values therebetween. In certain embodiments, the average particle size of the primary particles of sugar alcohol and/or saccharide is 30 μm or less, for example about 1-30 μm, about 5-30 μm, about 5-25 μm, about 5-20 μm, about 5-15 μm, about 5-10 μm, about 10-30 μm, about 10-25 μm, about 10-20 μm, about 10-15 μm, about 15-30 μm, about 15-25 μm, about 15-20 μm, about 20-30 μm, about 20-25 μm, or about 25-30 μm, or any other value or range of values therein.

Prior to coating with the taste-masking layer, the topiramate particles (e.g., crystalline or amorphous topiramate, granulated topiramate, or topiramate-layered beads) generally have an average particle size of about 1-100 μm, in some embodiments about 1-50 μm or about 1-30 μm, or average particle sizes as disclosed elsewhere herein. After coating with a taste-masking layer, the taste-masked topiramate-containing particles generally have an average particle size of less than about 500 μm. If the average particle size is significantly greater than about 500 μm, a disintegrated ODT comprising such particles can have an unpleasant “gritty” texture in the mouth of the patient, and other measures may need to be taken to increase palatability. When the average particle size is less than about 400 μm, the disintegrated ODT generally has a more palatable, “creamy” texture in the mouth of the patient.

The amount of rapidly dispersing granules or the amount of disintegrant-sugar alcohol/saccharide combination in relation to the taste-masked topiramate-containing particles can vary depending upon a desired disintegration rate and the desired organoleptic properties including taste-masking, mouthfeel and aftertaste. The amount of the disintegrant-sugar alcohol/saccharide combination in the compositions of the invention can range from about 40% to about 95%, including about 40%, about 45%, about 50%, about 55%, about 60%, about 65%, about 70%, about 75%, about 80%, about 85%, about 90%, and about 95%, inclusive of all values, ranges, and subranges there between. In one embodiment, the amount of disintegrant-sugar alcohol/saccharide combination is about 60-70% of the total weight of the composition. In another embodiment, the amount of disintegrant-sugar alcohol/saccharide combination is about 65% by weight.

The ODT compositions described herein contain a sufficient quantity of taste-masked drug-containing particles to provide a therapeutically effective dose of topiramate

The amount of the component drug in the ODT compositions described herein can range from about 2% to about 25%, including about 5%, about 10%, about 15%, about 20%, and about 25%, inclusive of all values, ranges, and subranges there between.

In addition to acceptable disintegration and organoleptic properties, commercially acceptable ODT formulations typically require hardness and friability suitable for packaging in bottles or in push-through film-backed and/or peel-off paper-backed blister packs for storage, transportation and commercial distribution. Accordingly, in addition to the taste-masked topiramate-containing particles, disintegrant, and sugar alcohol and/or saccharide, the ODT compositions described herein may also include other pharmaceutically acceptable ingredients or excipients which can aid in forming tablets with acceptable hardness and friability characteristics, promote rapid disintegration, and/or improve the organoleptic properties of the ODT formulations.

Examples of suitable excipients for use in the compositions or dosage forms described herein may include fillers, diluents, glidants, disintegrants, binders, lubricants etc. Other pharmaceutically acceptable excipients may include acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents, etc.

Examples of suitable fillers, diluents and/or binders may include lactose (e.g. spray-dried lactose, α-lactose, β-lactose, Tabletose®, various grades of Pharmatose®, Microtose® or Fast-Floc®), microcrystalline cellulose (e.g. Avicel PH101, Avicel PH102, Ceolus KG-802, Ceolus KG-1000, Prosolv SMCC 50 or SMCC90, various grades of Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g., basic calcium phosphate, calcium hydrogen phosphate, dicalcium phosphate hydrate), calcium sulfate, calcium carbonate, sodium alginate, collagen etc.

Specific examples of diluents may include calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Specific examples of glidants and lubricants may include stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients may include, e.g., flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Non-limiting examples of flavoring agents may include cherry, mint, orange, or other acceptable fruit flavors, and mixtures thereof at up to, for instance, about 3% based on the tablet weight. In addition, the compositions of the invention can also include one or more sweeteners such as aspartame, sucralose, or other pharmaceutically acceptable sweeteners, or mixtures thereof, at up to about 2% by weight, based on the tablet weight. Furthermore, the compositions of the invention can include one or more colorants, FD&C Red #7 (calcium lake), FD&C Red #27 (aluminum lake), FD&C Blue #1 (aluminum lake), at up to 0.5% by weight, based on the tablet weight.

Antioxidants may include, e.g., ascorbic acid, ascorbyl palmitate, butylated hydroxyanisole (BHA), butylated hydroxytoluene (BHT), hypophosphorous acid, monothioglycerol, potassium metabisulfite, propyl gallate, sodium formaldehyde sulfoxylate, sodium metabisulfite, sodium thiosulfate, sulfur dioxide, tocopherol, tocopherol acetate, tocopherol hemisuccinate, TPGS or other tocopherol derivatives, etc.

For example, the ODT compositions described herein can include a synthetic sweetener such as sucralose, a flavoring agent such as a cherry flavor, a tableting aide such as microcrystalline cellulose, and an additional disintegrant.

When the ODT compositions described herein include rapidly dispersing microgranules, the compositions can also include an additional disintegrant. The additional disintegrant can be the same disintegrant used in the rapidly dispersing microgranules, or a different disintegrant. The additional disintegrant may be present in the ODT compositions of the invention at up to about 10% based on the tablet weight, for example up to about 1%, up to about 2%, up to about 3%, up to about 4%, up to about 5%, up to about 6%, to about 7%, to about 8%, to about 9%, or to about 10%, including all ranges and subranges therebetween.

Specific examples of additional disintegrants may include, e.g., alginic acid or alginates, microcrystalline cellulose, hydroxypropyl cellulose and other cellulose derivatives, croscarmellose sodium, crospovidone, polacrilin potassium, sodium starch glycolate, starch, pregelatinized starch, carboxymethyl starch (e.g. Primogel® and Explotab®) etc. Specific examples of binders may include, e.g., acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch, etc.

In another embodiment, the ODT compositions described herein may comprise about 25-55% of topiramate crystals, microencapsulated with a taste-masking layer comprising a water-insoluble polymer (e.g., ethylcellulose); about 80-40% of rapidly-dispersing granules (e.g., comprising crospovidone and mannitol); about 5% of an additional disintegrant (e.g., crospovidone); about 1% of one or more flavors, and about 0.5%-1% of a sweetener (e.g., sucralose).

The ODT compositions described herein may comprise a therapeutically effective amount of topiramate coated with a taste-masking layer, e.g., in the form of a tablet further comprising rapidly dispersing granules comprising a disintegrant and a sugar alcohol and/or saccharide. Upon administration, the rapidly dispersing granules of the ODT tablet of the invention rapidly swell and/or dissolve in the patient's oral cavity, thereby causing disintegration of the ODT tablet into taste-masked, topiramate-containing particles to form a smooth, palatable, easy-to-swallow suspension.

In another embodiment, the ODT compositions described herein may comprise taste-masked topiramate-containing microparticles, one or more flavoring agents, a sweetener, rapidly-dispersing microgranules, microcrystalline cellulose, an additional disintegrant, and a lubricant such as magnesium stearate, compressed into orally disintegrating tablets. The orally disintegrating tablets formed thereby rapidly disintegrate on contact with saliva in the buccal cavity, and have a pleasant taste, a “creamy” mouth feel, and typically provide rapid, substantially-complete release of the topiramate dose in the stomach without causing significant gastric irritation.

In yet another embodiment, the ODT compositions described herein may comprise taste-masked drug microparticles and optionally flavoring agents, sweeteners, and other pharmaceutically acceptable excipients in a tablet press equipped with an externally lubricating system to pre-lubricate dies and punches, thereby providing an ODT formulation otherwise free of lubricant. The orally disintegrating tablets thus produced typically exhibit sufficient hardness and sufficiently low friability to be suitable for packaging in high-density polyethylene (HDPE) bottles and push-through film-backed or peel-off paper backed blister packs or alu-alu pouches, using conventional equipment for storage, transportation and commercial distribution. The optional flavoring agents, sweeteners, and other pharmaceutically acceptable excipients, tablet presses, etc., as well as compression conditions include, for example those described in U.S. Patent Application Publication Nos. US 2009/0202630, US 2009/0155360, US 2009/0169620, US 2009/0092672, US 2007/0196491, US 2007/0190145, US 2006/0105039, US 2006/0105038, US 2006/0078614, US 2006/0057199, and US 20050232988, each of which is hereby incorporated by reference in its entirety for all purposes.

The rate of disintegration of the ODT compositions described herein in the oral cavity of a patient can be on the order of about 60 seconds or less, about 50 seconds or less, about 40 seconds or less, about 30 seconds or less, about 20 seconds or less, or about 10 seconds or less (including all ranges and subranges therebetween).

The rate of disintegration can also be measured using various in vitro test methods, for example, the USP <701> Disintegration Test. When using the USP <701> Disintegration Test, the rates of disintegration of the present ODT compositions described herein are generally faster than those of conventional, non-ODT immediate release topiramate-containing compositions, for example, 60 seconds or less, 30 seconds or less, 20 seconds or less, or 10 seconds or 5 seconds or less. The term “non-ODT immediate release topiramate-containing compositions” refers to conventional tablets or capsules intended to be swallowed and absorb in the gastrointestinal tract, or chewable tablets which require mastication to break apart the tablet structure, and which do not contain extended release or controlled release coatings to delay the release of topiramate.

The dissolution rate of the ODTs described herein can be evaluated using the United States Pharmacopeia Apparatus 2 (paddles @ 50 rpm in 900 mL of 0.01 or 0.1N HCl, pH 3.0 or 4.5 or 6.8 (phosphate) buffer, or purified Water. When using the United States Pharmacopeia Apparatus 2, the rate of dissolution of the drug (e.g., topiramate) is comparable to that of conventional, non-ODT immediate release topiramate-containing compositions, for example, about 70%, about 75%, about 80%, about 85%, about 90%, about 95%, or about 100% of the topiramate is released in 30 min. In other words, the comparative dissolution rates for both test and reference formulations in relevant dissolution media are similar.

The present ODT compositions described herein provide good taste-masking when placed in the mouth until swallowed (e.g., not more than about 10% of the drug dose released in about 3 minutes when tested for dissolution in simulated saliva fluid at a pH of about 7.0). An ODT as described herein will typically disintegrate in about 30 seconds when evaluated using the USP <701> Disintegration Test, and will typically disintegrate on contact with saliva in the buccal cavity in vivo within about 60 seconds, forming a smooth, easy-to swallow suspension of taste-masked microparticles with an acceptable aftertaste. These taste-masked microparticles will typically provide substantially complete release of the topiramate dose contained in the ODT upon entering the stomach (e.g., not less than about 50%, or not less than about 60% of the topiramate dose is released in about 30 minutes when tested for dissolution in simulated gastric fluid or 0.01N HCl).

Drug-containing particles (e.g. topiramate-containing particles) according to embodiments of the present invention can be prepared by any suitable method. For example, the present drug-containing particles can be prepared by the granulation of drug crystals, one or more disintegrants, and one or more fillers (e.g., sugar alcohol, saccharide and/or microcrystalline cellulose) in a high shear granulator or a fluid-bed granulator using a solution of one or more polymeric binders, and dried in fluid bed equipment or on trays in a conventional oven to produce, e.g., topiramate-containing granules.

Alternatively, drug-containing particles can be prepared by layering a solution of the drug and a polymeric binder, dispersed or dissolved in a pharmaceutically acceptable solvent (e.g., water, alcohols such as ethanol, ketones such as acetone, hydrocarbons such as cyclohexane, and combinations thereof), onto an inert core (e.g., sugar beads, cellulose beads, or silica beads) e.g., in a fluid bed coating apparatus.

In certain embodiments of the invention, topiramate-containing particles (e.g., topiramate-containing granules, topiramate crystals and/or topiramate-layered beads) may be coated with a taste-masking layer by solvent coacervation or microencapsulation by phase separation with a water-insoluble polymer, or a combination of a water-insoluble polymer and a gastrosoluble pore-former (e.g., calcium carbonate) by the method described in U.S. patent application Ser. No. 11/213,266, which is herein incorporated by reference in its entirety for all purposes. For example, in one embodiment, topiramate may be layered on sugar spheres in a fluid-bed granulator and provided with a protective seal-coat (e.g., Opadry Clear). The resulting topiramate layered beads may then taste-masked by microencapsulation (e.g., by phase separation) in cyclohexane with a water-insoluble polymer (e.g., ethylcellulose) in combination with a gastrosoluble pore-former (e.g., calcium carbonate) to provide taste-masked beads as described in U.S. patent application Ser. No. 11/256,653, which is herein incorporated by reference for all purposes. Alternatively, topiramate (and/or one or more other drug) crystals with an average particle size range of about 1-200 μm, about 50-150 μm, or any other values or ranges of values therein, can be coated with a taste-masking layer by either fluid-bed coating or solvent coacervation in accordance with the present invention. Crystalline topiramate with a mean particle size of about 5-50 μm can also be taste-masked by solvent coacervation as described herein.

In certain other embodiments of the invention, topiramate-containing particles (e.g., topiramate-containing granules, topiramate crystals and/or topiramate-layered beads) may be coated with a taste-masking layer by fluid-bed coating with an enterosoluble polymer, or a combination of a water-insoluble polymer and an enterosoluble polymer. The enterosoluble polymer may be selected from the group consisting of hydroxypropyl methylcellulose or hypromellose phthalate, cellulose acetate phthalate, polyvinyl acetate phthalate, pH-sensitive methylmethacrylate copolymers commercially available as Eudragit L or S polymers, or combinations thereof.

Alcohol and/or drug abuse and dependence are widespread and it is estimated that about 20-25 million American adults abused alcohol and/or drugs or were dependent thereon in 2000. Research on the neurophysiology of alcohol and drug dependency and abuse indicates that alcohol and drug dependency and abuse may be related to alcohol- or drug-mediated stimulation of reward pathways in the brain due increased dopamine levels associated with the ingestion of alcohol and/or other drugs. Johnson and colleagues (Johnson, B. A. et al. (2003). “Oral topiramate for treatment of alcohol dependence: a randomized controlled trial”. The Lancet 361: 1677-1685; Johnson, B. A.; Rosenthal, N.; Capece, J. A.; Wiegand, F.; Mao, L.; Beyers, K.; McKay, A.; Ait-daoud, N. et al. (October 2007). “Topiramate for treating alcohol dependence: a randomized controlled trial”. Journal Amer. Med. Assoc. 298 (14): 1641-1651) postulated that topiramate might effectively treat alcohol dependence through several mechanisms, including the ability to decrease extracellular release of dopamine in the midbrain and antagonism of glutamate activity at the neuroreceptors. Most pharmacotherapy trials on alcoholism and/or drug addiction have focused on treatments with single pharmacological agents. However, studies with opioids such as naltrexone demonstrated the significance of opioid μ (e.g., beta endorphin), dopamine, serotonin (5-HT), γ-amino-butyric acid (GABA), and glutamate receptors for the development and maintenance of alcohol dependence. Johnson and colleagues (WO 2009/029308) investigated the use of combinations of drugs such as topiramate, ondansetron, and naltrexone that target multiple neurotransmitter systems for the treatment of alcoholism and drug addiction.

Appetite suppressors such as phentermine have been extensively used with or without prescriptions for achieving/maintaining weight loss. US Pat. Appl. Pub. No. 2009/0054372 discloses methods and kits comprising topiramate, phentermine, and vitamin B-12 for achieving or maintaining weight loss. For the treatment of alcoholism, drug addiction, as well as achieving/maintaining weight loss, it is most desirable if dosage forms comprising the required combinations of drugs are available as patient-friendly, convenient, and compliant, orally disintegrating tablets.

US Pat. Appl. Pub. No. 2009/0054372 (the contents of which are herein incorporated by reference in its entirety for all purposes) discloses a method and kits comprising therapeutically effective amounts topiramate, phentermine, and vitamin B-12 (e.g., cyanocobalamin) for achieving or maintaining weight loss. PCT Pat. Appl. Pub. No. WO 2009/029308 (the contents of which are herein incorporated by reference in its entirety for all purposes) discloses medication combinations comprising therapeutically effective amounts topiramate, naltrexone, and/or ondansetron for the treatment of alcoholism and drug addiction.

Accordingly, ODT compositions as described herein may also optionally include one or more additional active pharmaceutical ingredients suitable for treating the conditions described herein, and which are individually taste-masked. For example, a combination ODT product for achieving and/or maintaining weight loss may be prepared by blending taste-masked topiramate-containing particles, taste-masked phentermine-containing particles, and optionally taste-masked B-12 vitamin particles, rapidly dispersing microgranules, and other ODT excipients and compressing into orally disintegrating tablets. Phentermine-, naltrexone-, and/or ondansetron-containing particles (e.g., crystals, granules or drug layered beads thereof) may be taste-masked by coacervation with a water-insoluble polymer (e.g., ethylcellulose), by fluid bed coating with a water-insoluble polymer, and may include a fatty acid ester or may be in combination with a water-soluble, gastrosoluble or enterosoluble pore-former. A minor component of a combination ODT product, e.g., vitamin B-12, may be optionally granulated along with the components of rapidly dispersing granules to achieve both taste-masking and uniform dispersion in an ODT.

The drug-containing particles (e.g., drug-containing granules, drug crystals and/or drug-layered beads) may be taste-masked by, e.g., fluid-bed coating with a water-insoluble polymer in combination with a gastrosoluble polymer such as Eudragit E100 or EPO (an aminoalkyl methacrylate copolymer) by the method described in U.S. patent application Ser. No. 11/248,596, which is herein incorporated by reference in its entirety for all purposes.

For example, dissolved or suspended drug (e.g., topiramate) in a polymeric binder solution is layered onto inert particles (50-100 mesh or about 150-300 μm in diameter) such as sugar spheres or cellulose spheres (e.g., Celphere® CP-203) using a fluid-bed coater equipped with a bottom-spray Wurster insert. These drug-layered beads can then be taste-masked by fluid-bed coating or by solvent coacervation as described herein.

In a specific embodiment, a water-insoluble polymer (e.g., ethylcellulose), a phase-inducer (e.g., polyethylene), and topiramate are loaded into a coacervation tank containing cyclohexane. The mixture in the tank is heated to about 80° C. to dissolve the ethylcellulose, and then slowly cooled under controlled conditions thereby causing phase-induced microencapsulation of the topiramate-containing particles with the ethylcellulose. Upon reaching ambient temperature, the suspension of microencapsulated topiramate-containing particles is filtered, washed with fresh cyclohexane and dried to reduce residual solvent levels to within acceptable limits (e.g., less than about 4,000 ppm). In one embodiment, the residual solvent is present at a level of less than about 1,000 ppm. The coating weight of the microencapsulated topiramate-containing particles can range from about 3% to about 30% including about 5%, 10%, 15%, 20%, and 25%, inclusive of all ranges, subranges and values therein and therebetween. Examples of such a coacervation process are disclosed in U.S. Pat. Nos. 5,252,337, 5,639,475, 6,139,865 and 6,495,160, each of which is herein incorporated by reference in its entirety for all purposes.

Alternatively, a coacervation solution can comprise a mixture of a water-insoluble polymer (e.g., ethylcellulose) and a water-insoluble or gastrosoluble pore-former (e.g., calcium carbonate). The ratio of water-insoluble polymer to pore-former can range from about 50/50 to 95/05, including about 55/45, about 60/40, about 65/35, about 70/30, about 75/25, about 80/20, about 85/15, and about 90/10, including all ranges, subranges and values therein and there between. The coating weight of the microencapsulated drug particles can range from about 3% to about 20% including about 5%, 7.5%, 10%, 12.5%, 15%, and 17.5%, inclusive of all ranges, subranges and values therein and there between. In one embodiment, the coacervation step comprises suspending the topiramate-containing particles in a solution of ethylcellulose at about 80° C. in a coacervation tank. During the cooling cycle, a micronized pore-former is introduced into the tank at a temperature of about 58° C., while constantly stirring the suspension to uniformly distribute the pore-former in the microcapsule-membrane during a forming/hardening phase. Examples of such a coacervation process are disclosed in U.S. patent application Ser. No. 11/213,266.

In one embodiment, the ODT composition described herein may be prepared by a method comprising (a) granulating topiramate, e.g., with a filler and/or diluent such as a sugar alcohol and/or saccharide, (h) coating the topiramate-containing granules with a taste-masking layer, e.g., a water insoluble polymer such as ethylcellulose, by fluid bed coating or coacervation, (c) optionally preparing rapidly dispersing microgranules comprising a disintegrant such as crospovidone and a sugar alcohol such as mannitol with a mean primary particle size of not more than 30 μm in a high shear granulator (e.g., as described in U.S. patent application Ser. No. 10/356,641, which is herein incorporated by reference for all purposes), (d) blending the taste-masked topiramate granules with the rapidly dispersing granules, and optionally other pharmaceutically acceptable excipients, and (e) compressing the blend into an ODT composition.

In yet another embodiment, the ODT compositions described herein may be prepared by a method comprising (a) coating a solution or dispersion of topiramate and a pharmaceutically acceptable binder in a pharmaceutically acceptable solvent onto an inert core and removing the solvent to form a topiramate-layered bead, (b) coating the topiramate-layered beads with a taste-masking layer comprising water-insoluble ethylcellulose in combination with an enterosoluble polymer by fluid bed coating, (c) blending the taste-masked topiramate-layered beads with rapidly dispersing microgranules, and optionally other pharmaceutically acceptable excipients, and (d) compressing the blend into an ODT composition.

In still another embodiment, the ODT compositions described herein may be prepared by a method comprising (a) coating a solution or dispersion of topiramate and a pharmaceutically acceptable binder in a pharmaceutically acceptable solvent onto an inert core and removing the solvent to form a topiramate-layered bead, (b) coating the topiramate-layered beads with a taste-masking layer (e.g., ethylcellulose and a gastrosoluble pore-former, calcium carbonate) by fluid bed coating or coacervation, (c) coating granules of a disintegrant and a sugar alcohol and/or saccharide to form rapidly-disintegrating granules, (d) blending the taste-masked topiramate-layered beads and the rapidly-disintegrating granules, and optionally other pharmaceutically acceptable excipients, and (e) compressing the blend into the ODT composition. The taste-masked topiramate particles may comprise an appearance-masking coating layer disposed optionally under the taste-masking layer or over the layer, thereby making it difficult to distinctly visualizing the taste-masked topiramate in the ODT tablet matrix comprising rapidly dispersing granules and other pharmaceutically acceptable excipients.

In a particular embodiment, the ODT composition described herein may be prepared by (a) preparing topiramate-containing particles (e.g., by granulating topiramate crystalline material having an average particle size of about 5-50 μm and one or more diluents/fillers such as lactose, mannitol, microcrystalline cellulose and mixtures thereof, with a polymeric binder in a high-shear granulator or a fluid-bed coater, or topiramate-layered beads by dissolving the topiramate in a polymer binder solution and spraying the topiramate solution onto inert spheres (e.g., sugar spheres or cellulose spheres) in a fluid bed coater and applying a protective seal-coat); (b) taste-masking the topiramate-containing particles by microencapsulation (coacervation) or fluid bed coating with ethylcellulose alone or in combination with a gastrosoluble calcium carbonate or by fluid bed coating with ethylcellulose and Eudragit E100; (c) granulating one or more sugar alcohols and/or saccharides, each having an average particle diameter of not more than about 30 μm, with a disintegrant such as crospovidone, using water or an alcohol-water mixture in a conventional granulator, and drying the granulate in fluid-bed equipment or a conventional oven to produce rapidly-dispersing microgranules with an average particle size of not more than about 400 μm; (d) blending the taste-masked drug microparticles of step (b) with one or more flavoring agents, a sweetener, microcrystalline cellulose, additional disintegrant, and the rapidly-dispersing microgranules of step (c); and (e) compressing the blend of step (d) into the ODT composition. The compressing may be carried out using, e.g., a conventional rotary tablet press equipped with an external lubrication system to pre-lubricate the dies and punches. Examples of such a process of pre-lubricating the die and punch surfaces are disclosed in U.S. Pat. Nos. 5,996,902, 6,325,525, and 6,776,361, each of which is herein incorporated by reference in its entirety for all purposes.

The rapidly dispersing granules described herein can be prepared by any suitable method. For example, the rapidly dispersing granules can be prepared by granulation of one or more disintegrants and one or more sugar alcohols and/or saccharides in a high shear granulator, and dried in fluid bed equipment or on trays in a conventional oven to produce the rapidly dispersing granules. Rapidly-dispersing microgranules can also be produced by the method described in U.S. patent application Ser. No. 10/827,106, which is herein incorporated by reference in its entirety for all purposes.

In a particular embodiment, the ODT compositions described herein may be prepared by blending (a) topiramate-containing particles (e.g., topiramate-containing granules, topiramate crystals and/or topiramate-layered beads) taste-masked by any of the methods described in U.S. patent application Ser. Nos. 10/827,106; 11/213,266; 11/248,596; 11/256,653, each of which is herein incorporated by reference in its entirety; (b) rapidly dispersing microgranules that can be prepared by the method described in U.S. patent application Ser. No. 10/827,106, and (c) blending the topiramate-containing particles, rapidly dispersing granules, and other pharmaceutically acceptable ingredients such as a flavoring agent, a sweetener, a colorant, an additional disintegrant, and/or a compression aide such as microcrystalline cellulose, and (d) compressing the mixture into the ODT composition. The compressing step may be carried out using a rotary tablet press equipped with an external lubrication system to lubricate die and punch surfaces prior to compression.

### EXAMPLES

Embodiments of the present invention are described in greater detail in the sections below with reference to the examples. The following examples involving topiramate are used to illustrate the invention. It should be understood that the examples and embodiments described herein are for illustrative purposes only and that various modifications or changes in light thereof will be suggested to persons skilled in the art and are to be included within the spirit and purview of this application and the appended claims.

### Example 1

### 1.A Topiramate Microcapsules (Coating: 20% by Weight)

A 5-gallon tank equipped with a propeller mixer is charged with 10 kg cyclohexane, 800 g topiramate maleate, 200 g ethylcellulose (Ethocel™ Standard 100 Premium from Dow Chemical Company; EC-100) and 100 g Polyethylene (Epolene C-10 Wax). The tank is heated to about 80° C. while stirring the contents of the tank at 150 RPM. Once the temperature reaches 79-80° C., the tank is subjected to a controlled rate of cooling. Upon reaching <30° C., microcapsules that are formed are filtered and rinsed with fresh cyclohexane and allowed to dry overnight in the hood. Using the same procedure but with the use of different amounts of topiramate maleate, ethylcellulose and polyethylene, topiramate microcapsules with 10 wt. % and 15 wt. % ethylcellulose coating are also produced.

### 1.B Rapidly Dispersing Microgranules (95/5 Mannitol/Crospovidone)

D-mannitol (152 kg), a sugar alcohol with an average particle size of about 15 μm, and Crospovidone XL-10 (8 kg), a disintegrant, are mixed at a ratio of about 95/5 in a high shear granulator using purified water as the granulating fluid and dried in a fluid bed dryer to produce rapidly dispersing granules (PE278). Alternatively, the wet granules may be dried by spreading on trays in a heated convection oven (PE375).

### 1.C Microcapsules of Topiramate Granules (Drug Load: 85.0%).

Hydroxypropylcellulose (Klucel LF, 60.0 g) is slowly added to an ethanol (190 proof)-water mixture to dissolve. Topiramate with a mean particle size of <20 μm (1020.0 g) and hydrous lactose (120 g) are charged into Glatt GPCG 3 fluid-bed coater equipped with a top spray Wurster insert “C” air distribution plate; 100 mesh product retention screen and granulated by spraying the binder solution. The flow rate (e.g., 8-25 mL/min) is progressively increased while maintaining the product temperature (e.g., 40° C.) and the air volume (e.g., 40-55 CFM). Following the completion of spraying, the resulting granules are dried in the Glatt unit to drive off residual moisture/solvent.

Topiramate granules are taste-masked with ethylcellulose (EC-100) for a coating of 15 wt. % following the procedure of Example 1.A above.

### 1.D Topiramate ODTs, 100 and 200 mg

Topiramate taste-masked granules (120 mg equivalent to 100-mg of topiramate per 100 mg tablet) from Ex. 1.A above, rapidly dispersing granules prepared as described in Ex. 1.B (213 mg), and a pre-mix consisting of crospovidone (XL-10: ˜20 mg), microcrystalline cellulose (Avicel PH 101 from FMC Biopolymers; 40 mg), sucralose (˜1.4 mg), FD&C Red #7 (˜1.6 mg) and Mint flavor (˜4 mg) are blended for 3 min and compressed into ODTs using externally lubricated 13 mm round flat radius-edge punches/dies. Similarly, 200 mg (i.e., equivalent to 200 mg topiramate) ODT tablets are prepared having double the weight of the 100 mg equivalent topiramate tablets). The tablets are shown to meet the disintegration time specification of less than 30 seconds when tested by USP disintegration time test method <701> and release about 80% of the topiramate contained therein in 30 min when dissolution tested in 900 mL of 0.1N HCl at a paddle speed of 50 RPM.

### Example 2

### 2.A Topiramate Layered Beads (Drug Load: 45%)

A Glatt GPCG 5 fluid bed coater equipped with a 10″ Wurster insert, 16 mm tubing, 1 inch column gap, D air distribution plate, 200 mesh product retention screen, port size: 1.0 mm, nozzle cap: flush is charged with 60-80 mesh sugar spheres (2331 g). Topiramate (2250 g) is slowly added to an aqueous-organic solvent mixture to dissolve while constantly stirring for 30 min. Then hydroxypropylcellulose (Klucel LF, 169 g) is slowly added to the same solution to dissolve. The sugar spheres are coated by spraying at the following conditions: atomization air pressure: 2.5 bar; Air inlet temperature: 60° C.; product temperature: approximately 45° C. Air flow: 60 cfm; flow rate: 8 mL/min. Upon completion of drug layering, a 5 wt. % protective seal coat with Klucel LF is applied on the topiramate layered beads.

### 2.B Topiramate Taste-Masked Beads

A coacervation tank is charged with topiramate layered beads prepared as described above in Ex. 2.A, ethylcellulose, and polyethylene. The topiramate layered beads are microencapsulated at a 6.25% coating of ethylcellulose following the previously described procedure. The polyethylene content in the tank is varied from 0.5% to 2% to induce effects of the phase inducer.

2.C Topiramate Taste-Masked with EC-10/Eudragit EPO

The microencapsulated beads from Ex. 2.B above are further coated with ethylcellulose (EC-10) and Eudragit EPO polymer at a ratio of EC-10/EPO/TEC (triethyl citrate)/talc at 40/45/5/10 for a weight gain of about 15% by weight, as disclosed in the co-pending U.S. patent application Ser. No. 11/248,596 (published as US 2006/00614 A1) and dried in the same fluidized bed coater to drive off residual solvents. Following the same procedure, the 5 wt. % seal coated topiramate beads from Ex. 2.A above are taste-masked by fluid bed coating with EC-10/EPO/TEC/Mg stearate at a ratio of 42.5/42.5/5/10 for a weight gain of 20%.

### 2.D Topiramate ODT of Taste-Masked Beads

Topiramate taste-masked beads at a coating of 6.25% EC-100 followed by 15% EC-10/EPO from Ex. 2.0 above, rapidly dispersing microgranules prepared as disclosed in Ex. 1.B above, crospovidone (˜5%), microcrystalline cellulose (7-10% by weight), sucralose (0.35 wt. %, mint flavor (0.7%), and FD&C Red #27 (0.2%), and FD&C Blue #1(0.15% by weight) are blended in a V-blender for 15 min and compressed into ODTs (25, 50, 100, and 200 mg topiramate equivalent) as described in Ex. 1.D above. Following the same procedure, the 20% EC-10/EPO coated topiramate beads from Ex. 2.0 above are compressed into ODTs with dose strengths of 25, 50, 100 and 200 mg topiramate equivalent.

### Example 3

### 3.A Taste-Masked Topiramate Pellets Made in a Granurex

Povidone (PVP K-30) is slowly added to purified water while constantly stirring to prepare a polymer binder solution at 10% w/w solids. Topiramate micronized material is blended with colloidal silica (0.5% based on the weight of topiramate) a flow aid, Cab-O-Sil M-5P from Cabot Corporation) and povidone in a V-blender and charged into the product bowl of a Granurex GX-40 from Vector Corporation (Iowa, USA). The 10% PVP binder solution is sprayed into the rotating material bed at a controlled rate. Optimization parameters during forming pellets include—Process air temperature: ˜19-20° C.; Product temperature: 16±2° C.; Rotor speed: 425 RPM; External air supply: 150 L/min; Spray rate: 15 RPM (˜8 mL/min); pressure drop across slit: 1.3-11 mm in water; and during drying of pellets—Process air volume: 30 CFM; Process air temperature: ˜60° C.; Product temperature: 35° C. (to stop drying); rotor speed: 180 RPM; slit air volume: 10 CFM; processing time: 40 min. Pellets that are so prepared have about 65% of the particles in the size range of 50-100 mesh.

### 3.B Topiramate Taste-Masked by Phase Separation

Topiramate pellets with a drug load of 85 wt. % and a 5% seal coat with Opadry® White II, which are prepared following the procedures of Example 3.A above, ethylcellulose and Epolene C-10 are suspended in cyclohexane in a 5-gallon coacervation tank, and microcapsules at a coating of 15 wt. % are produced as disclosed previously. Using a procedure similar to that of Ex. 1.A, microcapsules with a 13 wt. % coating are also prepared by suspending topiramate pellets from Ex. 3.A above, in ethylcellulose and Epolene. Microcapsules with a coating of 17 wt. % are also prepared using a procedure similar to that of Ex. 1.A.

3.C Topiramate Taste-Masked with EC-10/EPO

Topiramate IR pellets with a drug load of 85 wt. % and a 5% seal coat with Opadry® Pink, which are prepared following the procedure of Example 3.A above, are coated with ethylcellulose (EC-10) and Eudragit EPO at a ratio of EC-10/EPO/TEC/Mg stearate at 42.5/42.5/5/10 for a coating of 25 wt. % in Glatt GPCG 5 following the procedure described in Ex. 2.0 above.

### 3.D Topiramate ODTs of Taste-Masked Granurex Pellets

Required amounts of taste-masked topiramate pellets (one part), rapidly dispersing microgranules (2 parts), crospovidone (5% by weight), microcrystalline cellulose (10 wt. %), mint flavor (0.6% by weight), and sucralose (0.35% by weight) are blended and compressed into ODTs (25-mg, 50-mg, 100-mg, and 200-mg) as described in Ex. 2.D above. A Hata production tablet press equipped with a vacuum transfer system, applicable tooling (round, flat face radius edge), tablet de-duster, a metal detector, and an externally lubricating Matsui ExLube system, is adjusted to provide tablets with a friability of less than 0.5% and adequate hardness values by varying the compression forces from about 5 kN to 16 kN. Magnesium stearate is used as a processing aid to externally lubricate the punch and die surfaces, and hence magnesium stearate may be present in trace amounts on the tablets. The weight range for each of the strengths is typically about +5% of the corresponding target tablet weight. The ExLube system is started to ensure that the lubricant is spraying properly when the tablet press is running. The tableting parameters such as fill depth (mm), pre-compression position (mm or kN) and main compression position (mm or kN) are adjusted on the press in order to produce tablets of each strength meeting set-up specifications. Following the successful set-up, the press is run in ‘Automatic Mode’ until completion. During the run, the tablets are sampled periodically to ensure that they meet appropriate in-process specifications.

### Example 4

### 4.A Phentermine Microcapsules

Phentermine hydrochloride particles are microencapsulated with ethylcellulose in cyclohexane following the procedures disclosed in Example 1.A above for a weight gain of 10, 15, and 20 wt. %. The dried microcapsules are sieved using appropriate sieves to discard oversized particles.

### 4.B Vitamin B-12 Granules

Vitamin B-12 (30 wt %), mannitol (60 wt. %), crospovidone (5%) and optionally mint flavor (1%) are granulated in Glatt GPCG 3 by spraying a hydroxypropylcellulose (4% Kluce LF) solution as disclosed in Ex. 1.C.

### 4.C Naltrexone Taste-Masking

Naltrexone hydrochloride (15% by weight), mannitol (78.4%), crospovidone, and optionally sucralose (0.6%) and cherry flavor (1%) are granulated in Glatt 3 by spraying a solution of Klucel LF (5% hydroxypropylcellulose) as disclosed in Ex. 4.B above.

### 4.D Ondansetron Taste-Masking

Ondansetron hydrochloride particles are microencapsulated with ethylcellulose in cyclohexane following the procedures disclosed in Example 1.A above for a weight gain of 10 wt. %. The dried microcapsules are sieved to discard oversized particles.

### 4.E Topiramate/Phentermine HCl/B-12 Vitamin ODTs

Required amounts of taste-masked topiramate based on the assay, taste-masked phentermine microcapsules, B-12 vitamin (cyanocobalamin) granules, rapidly dispersing microgranules (at least 2 parts to one part of taste-masked (topiramate+phentermine+B-12)), microcrystalline cellulose (10% Avicel PH101), crospovidone (5%), sucralose (0.5%), and cherry flavor (0.8%) are blended together to achieve homogeneity and compressed into ODTs comprising 15 mg of topiramate, 15 mg phentermine HCl, and 1 mg B-12 vitamin or 30 mg of topiramate, 30 mg phentermine, and 1 mg B-12 vitamin. The tablets thus produced are found to disintegrate rapidly in the oral cavity forming a smooth, easy-to-swallow suspension, and rapidly releasing the drugs upon ingestion to be bioequivalent/biosimilar to TOPOMAX (25 or 50 mg topiramate) and/or 37.5 mg phentermine.

### 4.F Topiramate/Ondansetron/Naltrexone HCl ODTs

Required amounts of taste-masked topiramate, taste-masked ondansetron beads, taste-masked naltrexone granules, rapidly dispersing microgranules (2 parts to one part of taste-masked (topiramate+ondansetron+naltrexone)), microcrystalline cellulose (10% Avicel PH101), crospovidone (5%), sucralose (0.5%), and cherry flavor (0.7%) are blended together to achieve blend homogeneity and compressed into ODTs comprising 25 mg of topiramate, 4 mg ondansetron, and 1 mg naltrexone or 50 mg of topiramate, 4 mg ondansetron, and 0.5 mg naltrexone. The tablets thus produced are found to disintegrate rapidly in the oral cavity forming a smooth, easy-to-swallow suspension, and rapidly releasing the drugs upon ingestion to be bioequivalent or biosimilar to TOPOMAX (25 or 50 mg topiramate) and/or Zofran (4 mg ondansetron).

While the present invention has been described in connection with certain embodiments described herein, it will be understood that the present application and the appended claims are intended to cover any variations, uses, modifications, equivalents or adaptations of the various embodiments of the present invention following, in general, the principles of the invention and including such departures from the present disclosure as come within known or customary practice within the art to that the invention pertains and as may be applied to the essential features set forth herein and as follows in the scope of the appended claims.

What is claimed:

1. A taste masked pharmaceutical composition comprising a therapeutically effective amount of taste masked sulfamate-substituted monosaccharide particles comprising a sulfamate-substituted monosaccharide or a pharmaceutically acceptable salt or derivative thereof, wherein said particles are coated with one or more taste-masking layers to taste mask the sulfamate-substituted monosaccharide; wherein said taste-masking layer(s) comprise(s) at least one water-insoluble, polymer.

2. An orally disintegrating tablet comprising: (1) the taste masked pharmaceutical composition of claim 1, and (2) rapidly dispersing microgranules comprising at least one disintegrant, and at least one sugar alcohol and/or at least one saccharide.

3. The orally disintegrating tablet of claim 2, which substantially disintegrates within a patient's oral cavity within about 60 seconds after administration therein.

4. The orally disintegrating tablet of claim 2, which substantially disintegrates within about 30 seconds when tested by the USP <701> Disintegration Test.

5. The taste masked pharmaceutical composition of claim 1, wherein about 70% or more of said sulfamate-substituted monosaccharide or pharmaceutically acceptable salt or derivative thereof is released from said particles within about 30 minutes when tested for dissolution using United States Pharmacopeia Apparatus 2 paddles at 50 rpm in 900 mL of 0.1 N HCl.

6. The taste masked pharmaceutical composition of claim 1, wherein said sulfamate-substituted monosaccharide or a pharmaceutically acceptable salt or derivative thereof is topiramate or a pharmaceutically acceptable salt or derivative thereof.

7. The taste masked pharmaceutical composition of claim 1, wherein said sulfamate-substituted monosaccharide or a pharmaceutically acceptable salt or derivative thereof is topiramate, said particles have an average particle size of about 1-300 μm, and the taste masked topiramate particles have an average particle size of 400 μm or less.

8. The taste masked pharmaceutical composition of claim 1, wherein said particles are crystals, microgranules or drug-layered beads comprising an inert core coated with said sulfamate-substituted monosaccharide or a pharmaceutically acceptable salt or derivative thereof and a polymer binder.

9. The taste masked pharmaceutical composition of claim 1, wherein said taste-masking layer further comprises a water-soluble, gastrosoluble, or enterosoluble pore former.

10. The taste masked pharmaceutical composition of claim 1, comprising about 1 wt % to about 70 wt % said taste masked sulfamate-substituted monosaccharide particles.

11. The taste masked pharmaceutical composition of claim 1, wherein the taste masked sulfamate-substituted monosaccharide particles further comprise a protective seal coat comprising a hydrophilic polymer in an amount of from about 1 wt % to about 8 wt % of said particles.

12. The taste masked pharmaceutical composition of claim 1, wherein said water-insoluble polymer is selected from the group consisting of ethylcellulose, cellulose acetate, cellulose acetate butyrate, polyvinyl acetate, neutral methacrylic ester copolymer, ammonio methacrylate copolymers and mixtures thereof.

13. The taste masked pharmaceutical composition of claim 1, wherein said taste-masking layer further comprises a water-soluble pore former selected from the group consisting of povidone, lactose, sodium chloride, sucrose, methylcellulose, hydroxypropylcellulose, hydroxypropyl methylcellulose, polyethylene glycol, and mixtures thereof.

14. The taste masked pharmaceutical composition of claim 1, wherein said taste-masking layer further comprises a gastrosoluble pore former selected from the group consisting of calcium carbonate, magnesium oxide, aminoalkyl methacrylate copolymers, polyvinylacetal diethylaminoacetate, and mixtures thereof.

15. The taste masked pharmaceutical composition of claim 1, wherein said taste-masking layer further comprises an enterosoluble pore former selected from the group consisting of cellulose acetate phthalate, hypromellose phthalate, Eudragit® L100 or S100, and mixtures thereof.

16. The taste masked pharmaceutical composition of claim 9, wherein the water-insoluble taste-masking polymer in combination with a water-soluble, enterosoluble or gastrosoluble pore former has a ratio of water-insoluble polymer to water-soluble, enterosolublc, or gastrosoluble pore former ranging from about 90/10 to about 50/50.

17. The taste masked pharmaceutical composition of claim 1, wherein the water-insoluble polymer is ethylcellulose having a viscosity of about 10-100 cps when tested as a 5 wt % solution at about 23° C.

18. The orally disintegrating tablet of claim 2, wherein the at least one disintegrant and the at least one sugar alcohol and/or at least one saccharide are present at a ratio of sugar alcohol and/or saccharide to disintegrant of from about 90/10 to about 99/1.

19. The orally disintegrating tablet of claim 2, wherein the disintegrant is selected from the group consisting of crospovidone, sodium starch glycolate, crosslinked carboxymethyl cellulose of sodium, low-substituted hydroxypropylcellulose and mixtures thereof.

20. The orally disintegrating tablet of claim 2, wherein the sugar alcohol and/or saccharide is selected from the group consisting of mannitol, xylitol, sorbitol, maltitol, lactose, sucrose, maltose and mixtures thereof.

21. The taste masked pharmaceutical composition of claim 1, further comprising taste-masked drug-containing particles comprising an appetite suppressant of the amphetamine and/or phenylethylamine class.

22. The taste masked pharmaceutical composition of claim 21, further comprising taste-masked particles comprising vitamin B-12.

23. The taste masked pharmaceutical composition or orally disintegrating tablet of claim 22, comprising an effective amount of said taste-masked particles comprising topiramate, phentermine, and vitamin B-12.

24. The taste masked pharmaceutical composition of claim 1, further comprising taste-masked particles comprising a 5-HT₃ receptor antagonist.

25. The taste masked pharmaceutical composition of claim 24, further comprising taste-masked microparticles comprising an opioid receptor antagonist.

26. The taste masked pharmaceutical composition of claim 25, comprising an effective amount of said taste-masked microparticles comprising topiramate, a 5-HT₃ receptor antagonist, and an opioid receptor antagonist for the treatment of alcoholism or drug addiction, wherein said a 5-HT₃ receptor antagonist is ondansetron or a pharmaceutically acceptable salt thereof and said opioid receptor antagonist is naltrexone.

27. A method of preparing the orally disintegrating tablet of claim 2, comprising:
preparing said particles comprising sulfamate-substituted monosaccharide or a pharmaceutically acceptable salt or derivative thereof;
coating said particles with said taste-masking layer(s) to form said taste masked sulfamate-substituted monosaccharide particles;
mixing said taste masked sulfamate-substituted monosaccharide particles with said rapidly dispersing microgranules and optionally one or more pharmaceutically acceptable excipients; and
compressing the mixture to form said orally disintegrating tablet.

28. The method of claim 27, wherein preparing said particles comprises:
dissolving a sulfamate-substituted monosaccharide or a pharmaceutically acceptable salt or derivative thereof and a binder in a pharmaceutically acceptable solvent to form a sulfamate-substituted monosaccharide solution;
coating the sulfamate-substituted monosaccharide solution onto inert cores; and
evaporating the pharmaceutically acceptable solvent to form said microparticles.

29. The method of claim 28, wherein preparing said microparticles further comprises:
granulating a sulfamate-substituted monosaccharide or a pharmaceutically acceptable salt or derivative thereof with one or more pharmaceutically acceptable fillers and one or more polymeric binders to form said microparticles.

30. The method of claim 27, wherein coating comprises coacervation.

31. The method of claim 27, wherein coating comprises fluid bed coating.

32. The method of claim 27, wherein said compressing is effected using a rotary tablet press equipped with an external lubrication system to pre-lubricate the dies and punches.

33. The orally disintegrating tablet of claim 2, further comprising one or more pharmaceutically acceptable excipients comprising a flavoring agent and/or a sweetener.

35. A method of treating a patient subject to partial onset or primary generalized tonic-clonic seizures, seizures associated with Lennox-Gastaut syndrome, and/or dysphagia, comprising administering to the patient a pharmaceutically effective amount of the pharmaceutical composition of claim 2.
